(19) United States
(12) Patent Application Publication
(10) Pub. No.: US 2024/0272142 A1
(43) Pub. Date: Aug. 15, 2024

(54) METHOD FOR DETERMINING WHITENING EFFICACY OF COSMETIC RAW MATERIAL AND USE THEREOF

(71) Applicant: SHENZHEN INSTITUTE FOR DRUG CONTROL (SHENZHEN TESTING CENTER OF MEDICAL DEVICES), Shenzhen (CN)

(72) Inventors: Meirong Qin, Shenzhen (CN); Xiaowei Wang, Shenzhen (CN); Xiaoyu Feng, Shenzhen (CN); Zhihong Yao, Shenzhen (CN); Ping Wang, Shenzhen (CN); Pei Lin, Shenzhen (CN); Zhanlin Ma, Shenzhen (CN); Xiaoqiong Zheng, Shenzhen (CN)

(21) Appl. No.: 18/422,851

(22) Filed: Jan. 25, 2024

(30) Foreign Application Priority Data
Feb. 13, 2023 (CN) 202310105487.9

(51) Int. Cl. G01N 33/50; C12N 5/09

(52) U.S. Cl. CPC G01N 33/502; C12N 5/0693; C12N 2503/02

(57) ABSTRACT

Provided is a method for determining whitening efficacy of a cosmetic raw material. In the disclosure, specific binding of melanocyte-stimulating hormone (α-MSH) to melanocortical receptor I (MC1R) on melanocytes activates adenylyl cyclase (AC). The AC could catalyze the conversion of adenosine triphosphate (ATP) into cyclic adenosine monophosphate (cAMP), causing an increased level of intracellular cAMP. Increased CAMP level activates tyrosinase through protein kinase A (PKA), thereby promoting melanin production, and then achieving the determination of a relative melanin content.

### CROSS REFERENCE TO RELATED APPLICATION

This patent application claims the benefit and priority of Chinese Patent Application No. 202310105487.9 filed with the China National Intellectual Property Administration on Feb. 13, 2023, the disclosure of which is incorporated by reference herein in its entirety as part of the present application.

### TECHNICAL FIELD

The present disclosure relates to the technical field of cosmetic raw materials, and in particular to a method for determining whitening efficacy of a cosmetic raw material and use thereof.

### BACKGROUND

At present, the whitening efficacy of whitening cosmetic raw materials is generally determined through tyrosinase activity inhibition experiments. However, some studies have shown that not all cosmetic raw materials with whitening efficacy generally work by inhibiting tyrosinase activity. This results in the inability to comprehensively and accurately determine whether a cosmetic raw material has the whitening efficacy through the tyrosinase activity inhibition experiments. Moreover, mushroom tyrosinase in the tyrosinase activity inhibition experiments is extremely susceptible to the influence of environmental temperature, thus causing deviated experimental results. Therefore, it is one of the technical problems that urgently need to be solved to provide a rapid, accurate, and less environmentally-affected method for screening a cosmetic raw material with whitening efficacy.

### SUMMARY

In order to solve the above problems, the present disclosure provides a method for screening a cosmetic raw material with whitening efficacy and use thereof. In the present disclosure, a high-expression model in which melanocyte-stimulating hormone (α-MSH) induces mouse melanoma cells (B16-F10) to produce melanin is established at the cellular level, thereby achieving rapid and accurate determination of the whitening efficacy of the cosmetic raw material as well as efficient screening of the cosmetic raw material with the whitening efficacy.

The present disclosure provides a method for determining whitening efficacy of a cosmetic raw material, including the following steps:
(1) mixing α-melanocyte-stimulating hormone (α-MSH) with a phosphate-buffered saline (PBS) to obtain a 500 μM α-MSH solution;
(2) adding fetal bovine serum (FBS) and a double antibody into a high-glucose Dulbecco's modified eagle medium (DMEM) to obtain a complete DMEM, the FBS accounting for 10% of a total volume of the complete DMEM and the double antibody accounting for 1% of the total volume of the complete DMEM;
(3) inoculating a cell suspension of mouse melanoma (B16-F10) cells in a logarithmic growth phase into 6-well plates in an amount of 2 mL in each well, and culturing in a 5% carbon dioxide incubator at a temperature of 37° C. for 24 h;
(4) removing a medium contained in the 6-well plates, washing the B16-F10 cells with 2 mL of the PBS, adding 2 mL of a basal DMEM into each well of one of the 6-well plates to obtain a blank group, adding 1.8 mL of a sample to be tested and 200 μL of the 500 μM α-MSH solution into each well of another one of the 6-well plates to obtain an experimental group, and culturing the blank group and the experimental group in the 5% carbon dioxide incubator at a temperature of 37° C. for 48 h; and
(5) removing the basal DMEM in the 6-well plates by using a sterile pipette, washing the B16-F10 cells with 2 mL of the PBS, digesting the B16-F10 cells with 400 μL of trypsin, terminating the digesting with 1 mL of the complete DMEM, collecting the B16-F10 cells into centrifuge tubes, centrifuging the B16-F10 cells at 2,500 rpm for 10 min, discarding a resulting supernatant after centrifugation, centrifuging the B16-F10 cells with 1,000 μL of the PBS for a second time, adding 500 μL of a 1 mol/L NaOH solution into each of the centrifuge tubes, and conducting a water bath at a temperature of 80° C. for 1 h to obtain a product, transferring 200 μL of the product to a 96-well plate, determining an absorbance at 405 nm, and calculating to obtain a relative melanin content.

In some embodiments, the double antibody in step (2) is a penicillin-streptomycin double antibody.

In some embodiments, the inoculating in step (3) is conducted at a cell density of 2×10⁵ cells/mL.

In some embodiments, a concentration of the sample to be tested in step (4) is one or more selected from the group consisting of 0.5 μM, 1.0 μM, 2.5 μM, 5 μM, and 10 μM.

In some embodiments, the method further comprises providing a positive control group in step (4), and the positive control group is set by adding 1.8 mL of β-arbutin and 200 μL of an α-MSH solution into each well of further one of the 6-well plates.

In some embodiments, the method further comprises providing a model group in step (4), and the model group is set by adding 1.8 mL of the sample to be tested with different concentrations and 200 μL of the 500 μM α-MSH solution into each well of further one of the 6-well plates.

In some embodiments, the NaOH solution in step (5) includes 10% of dimethyl sulfoxide (DMSO).

In some embodiments, the NaOH solution including the 10% of DMSO is prepared by a process including: dissolving 1 g of NaOH in an appropriate amount of water, adding 2.5 mL of DMSO to obtain a mixed solution, diluting the mixed solution into a 25 mL volumetric flask with water, and mixing evenly.

In some embodiments, a calculation formula of the relative melanin content in step (5) is: the relative melanin content=absorbance of the experimental group/absorbance of the blank group×100%.

The present disclosure further provides use of the method for determining whitening efficacy of a cosmetic raw material in screening the cosmetic raw material with the whitening efficacy.

In the present disclosure, specific binding of melanocyte-stimulating hormone (α-MSH) to melanocortical receptor I (MC1R) on melanocytes activates adenylyl cyclase (AC). The AC could catalyze the conversion of adenosine triphosphate (ATP) into cyclic adenosine monophosphate (cAMP), causing an increased level of intracellular cAMP. Increased cAMP level activates tyrosinase through protein kinase A (PKA), thereby promoting melanin production, and then achieving the determination of a relative melanin content.

Compared with the prior art, some embodiments of the present disclosure have the following beneficial technical effects:

In the present disclosure, the method overcomes the problem that existing tyrosinase activity inhibition experiments are greatly affected by environmental factors, the high-expression model of melanin production in B16-F10 cells induced by α-MSH is established, thereby achieving rapid and accurate determination of the whitening efficacy of the cosmetic raw material as well as efficient screening of the cosmetic raw material with the whitening efficacy.

### BRIEF DESCRIPTION OF THE DRAWINGS

The present disclosure will be further described below in conjunction with drawings.

FIG. 1 shows a relative melanin content of a sample in Example 1 of the present disclosure after 24 h of stimulation;

FIG. 2 shows a relative melanin content of a sample in Example 2 of the present disclosure after 48 h of stimulation; and

FIG. 3 shows a relative melanin content of a positive control group stimulated with ß- arbutin at different concentrations for 48 h in Example 3 of the present disclosure.

### DETAILED DESCRIPTION OF THE EMBODIMENTS

The present disclosure provides a method for determining whitening efficacy of a cosmetic raw material, including the following steps:
(1) mixing α-melanocyte-stimulating hormone (α-MSH) with a phosphate-buffered saline (PBS) to obtain a 500 μM α-MSH solution;
(2) adding fetal bovine serum (FBS) and a double antibody into a high-glucose Dulbecco's modified eagle medium (DMEM) to obtain a complete DMEM, the FBS accounting for 10% of a total volume of the complete DMEM and the double antibody accounting for 1% of the total volume of the complete DMEM;
(3) inoculating a cell suspension of mouse melanoma (B16-F10) cells in a logarithmic growth phase into 6-well plates in an amount of 2 mL in each well, and culturing in a 5% carbon dioxide incubator at a temperature of 37° C. for 24 h;
(4) removing a medium contained in the 6-well plates, washing the B16-F10 cells with 2 mL of the PBS, adding 2 mL of a basal DMEM into each well of one of the 6-well plates to obtain a blank group, adding 1.8 mL of a sample to be tested and 200 μL of the 500 μM α-MSH solution into each well of another one of the 6-well plates to obtain an experimental group, and culturing the blank group and the experimental group in the 5% carbon dioxide incubator at a temperature of 37° C. for 48 h; and
(5) removing the basal DMEM in the 6-well plates by using a sterile pipette, washing the B16-F10 cells with 2 mL of the PBS, digesting the B16-F10 cells with 400 μL of trypsin, terminating the digesting with 1 mL of the complete DMEM, collecting the B16-F10 cells into centrifuge tubes, centrifuging the B16-F10 cells at 2,500 rpm for 10 min, discarding a resulting supernatant after centrifugation, centrifuging the B16-F10 cells with 1,000 μL of the PBS for a second time, adding 500 μL of a 1 mol/L NaOH solution into each of the centrifuge tubes, and conducting water bath at a temperature of 80° C. for 1 h to obtain a product,, transferring 200 μL of the product to a 96-well plate, determining an absorbance at 405 nm, and calculating to obtain a relative melanin content.

In an embodiment, the double antibody in step (2) is a penicillin-streptomycin double antibody.

In an embodiment, the inoculating in step (3) is conducted at a cell density of 2×10⁵ cells/mL.

In an embodiment, a concentration of the sample to be tested in step (4) is one or more selected from the group consisting of 0.5 μM, 1.0 μM, 2.5 μM, 5 μM, and 10 μM.

In an embodiment, the method further comprises providing a positive control group in step (4), and the positive control group is set by adding 1.8 mL of β-arbutin and 200 μL of an α-MSH solution into each well of further one of the multiple 6-well plates.

In an embodiment, the method further comprises providing a model group in step (4), and the model group is set by adding 1.8 mL of the sample to be tested with different concentrations and 200 μL of the 500 μM α-MSH solution into each well of further one of the multiple 6-well plates.

In an embodiment, the NaOH solution in step (5) includes 10% of dimethyl sulfoxide (DMSO).

In an embodiment, a calculation formula of the relative melanin content in step (5) is: the relative melanin content=absorbance of the experimental group/absorbance of the blank group×100%.

The present disclosure further provides use of the method for determining whitening efficacy of a cosmetic raw material in screening the cosmetic raw material with the whitening efficacy.

The technical solutions provided by the present disclosure will be further described below in conjunction with examples.

The results in the examples of the present disclosure are all expressed in terms of a relative melanin content: the relative melanin content=absorbance of the experimental group/absorbance of the blank group×100%.

### Experimental Reagents

Mouse melanoma cells B16-F10 (Guangzhou Cellcook Biotech Co., Ltd., China); high-glucose DMEM: (Gibco: 11995-065); PBS (pH=7.4, 1×; Gibco: 10010-023); fetal bovine serum (FBS; Gibco:10091-148); 0.25% Trypsin-EDTA (1×; Gibco: 25200-072); penicillin-streptomycin double antibody (PenicilinStreptomycin; Gibco: 15140-122); CCK8 kit (Beyotime (Shanghai) Biotechnology Co., Ltd., China); melanocyte-stimulating hormone (α-MSH; Shanghai Yuanye Biotechnology Co., Ltd., China); β-arbutin (National (China) Institutes for Food and Drug Control).

### Example 1

(1) A cell suspension of B16-F10 cells in a logarithmic growth phase was inoculated into 6-well plates in a cell density of 2×10⁵ cells/mL, with an amount being 2 mL in each well, and then added into a 5% carbon dioxide incubator and cultured at a temperature of 37° C. for 24 h.
(2) A medium contained in the 6-well plates was removed, the B16-F10 cells were washed with 2 mL of the PBS, 2 mL of substances from each of the following groups each were added into each well of the 6-well plates, and then cultured in the 5% carbon dioxide incubator at a temperature of 37° C. for 24 h.

Blank group: 2 mL of a basal DMEM was added into each well.

Model group: 2 mL of a-MSH solutions with different concentrations (0.5 μM, 1 μM, 2.5 μM, 5 μM, and 10 μM) were separately added into each well.
(3) Determination of a relative melanin content: the basal DMEM in the 6-well plates was removed by using a sterile pipette, the B16-F10 cells were washed with 2 mL of the PBS, and digested with 400 μL of trypsin; then the digesting was terminated with 1 mL of a complete DMEM; the B16-F10 cells were collected into centrifuge tubes, and centrifuged at 2,500 rpm for 10 min; a resulting supernatant after centrifugation was discarded, the resulting B16-F10 cells were centrifuged with 1,000 μL of the PBS for a second time, 500 μL of a 1 mol/L NaOH solution (containing 10% of DMSO) was added into each of the centrifuge tubes, and subjected to water bath at a temperature of 80° C. for 1 h to obtain a product; 200 μL of the product was transferred into a 96-well plate, an absorbance at 405 nm was determined and calculated to obtain the relative melanin content.

Experimental results are in the Table below:

| Sample name | Reaction concentration | Relative melanin content, % | | |
|---|---|---|---|---|
| BC | / | 98.99 | 100.17 | 100.84 |
| α-MSH | 0.5 μM | 102.51 | 111.76 | 97.21 |
| | 1.0 μM | 104.65 | 100.54 | 103.16 |
| | 2.5 μM | 98.75 | 98.22 | 102.18 |
| | 5.0 μM | 96.79 | 97.45 | 95.71 |
| | 10 μM | 111.71 | 108.05 | 112.79 |

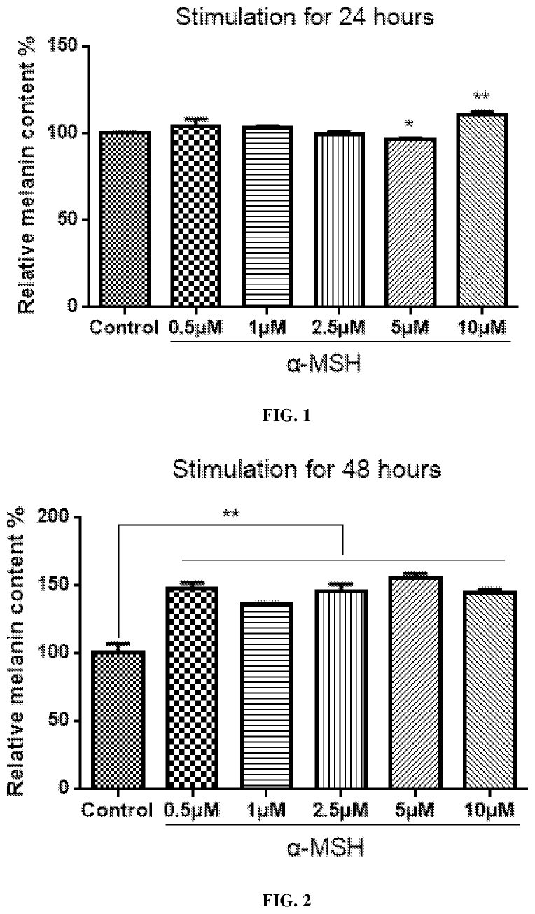

Results of Example 1 are shown in FIG. 1.

### Example 2

(1) A cell suspension of B16-F10 cells in a logarithmic growth phase was inoculated into 6-well plates in a cell density of 2×10⁵ cells/mL, with an amount being 2 mL in each well, and then added into a 5% carbon dioxide incubator and cultured at a temperature of 37° C. for 24 h;
(2) A medium contained in the 6-well plates was removed, the B16-F10 cells were washed with 2 mL of the PBS, 2 mL of substances from each of the following groups each were added into each well of the 6-well plates, and then cultured in the 5% carbon dioxide incubator at a temperature of 37° C. for 48 h.

Blank group: 2 mL of a basal DMEM was added into each well.

Model group: 2 mL of α-MSH solutions with different concentrations (0.5 μM, 1 μM, 2.5 μM, 5 μM, and 10 μM) were separately added into each well.
(3) Determination of a relative melanin content: the basal DMEM in the 6-well plates was removed by using a sterile pipette, the B16-F10 cells were washed with 2 mL of the PBS, and digested with 400 μL of trypsin, then the digesting was terminated with 1 mL of a complete DMEM, the B16-F10 cells were collected into centrifuge tubes, and centrifuged at 2,500 rpm for 10 min, a resulting supernatant after centrifugation was discarded, the resulting B16-F10 cells were centrifuged with 1,000 μL of the PBS for a second time, 500 μL of a 1 mol/L NaOH solution (containing 10% of DMSO) was added into each of the centrifuge tubes, and subjected to water bath at a temperature of 80° C. for 1 h to obtain a product; 200 μL of the product was transferred into a 96-well plate, an absorbance at 405 nm was determined and calculated to obtain the relative melanin content.

Results of Example 2 are shown in FIG. 2.

### Example 3

(1) A cell suspension of B16-F10 cells in a logarithmic growth phase was inoculated into 6-well plates in a cell density of 2×10⁵ cells/mL, with an amount being 2 mL in each well, and then added into a 5% carbon dioxide incubator and cultured at a temperature of 37° C. for 24 h.
(2) A medium contained in the 6-well plates was removed, the B16-F10 cells were washed with 2 mL of the PBS, 2 mL of substances from each of the following groups each were added into each well of the 6-well plates, and then cultured in the 5% carbon dioxide incubator at a temperature of 37° C. for 48 h.

Blank group: 2 mL of a basal DMEM was added into each well.

Model group: 2 mL of α-MSH solution with a concentration of 0.5 μM was added into each well;

Positive control group: 1.8 mL of β-arbutin with different concentrations and 200 μL of a 5 μM α-MSH solution (a final concentration after addition was 0.5 μM) were separately added into each well.
(3) Determination of a relative melanin content: the basal DMEM in the 6-well plates was removed by using a sterile pipette, the B16-F10 cells were washed with 2 mL of the PBS, and digested with 400 μL of trypsin, then the digesting was terminated with 1 mL of a complete DMEM, the B16-F10 cells were collected into centrifuge tubes, and centrifuged at 2,500 rpm for 10 min, a resulting supernatant after centrifugation was discarded, the resulting B16-F10 cells were centrifuged with 1,000 μL of the PBS for a second time, 500 μL of a 1 mol/L NaOH solution (containing 10% of DMSO) was added into each of the centrifuge tubes, and subjected to water bath at a temperature of 80° C. for 1 h to obtain a product, and 200 μL of the product was transferred into a 96-well plate; an absorbance at 405 nm was determined and calculated to obtain the relative melanin content.

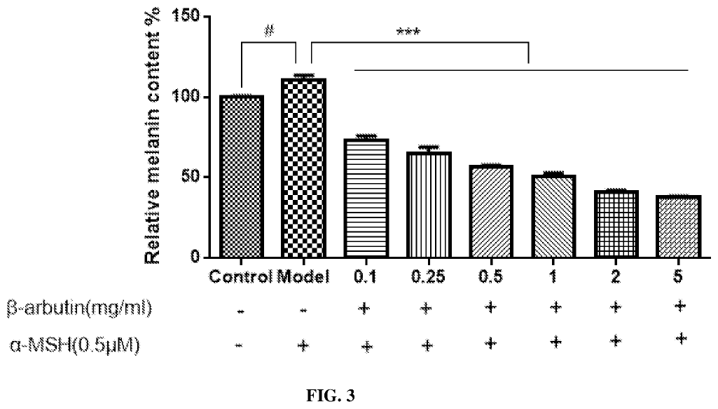

Results of Example 3 are shown in FIG. 3.

Specific examples are used herein for illustration of principles and embodiments of the present disclosure. The descriptions of the above embodiments are merely used for assisting in understanding the method of the present disclosure and its core ideas. In addition, those of ordinary skill in the art could make various modifications in terms of particular embodiments and the scope of application in accordance with the ideas of the present disclosure. In conclusion, the content of the description should not be construed as limitations to the present disclosure.

What is claimed is:

1. A method for determining whitening efficacy of a cosmetic raw material, comprising the following steps:
(1) mixing α-melanocyte-stimulating hormone (α-MSH) with a phosphate-buffered saline (PBS) to obtain a 500 μM α-MSH solution;
(2) adding fetal bovine serum (FBS) and a double antibody into a high-glucose Dulbecco's modified eagle medium (DMEM) to obtain a complete DMEM, the FBS accounting for 10% of a total volume of the complete DMEM and the double antibody accounting for 1% of the total volume of the complete DMEM;
(3) inoculating a cell suspension of mouse melanoma (B16-F10) cells in a logarithmic growth phase into 6-well plates in an amount of 2 mL in each well, and culturing in a 5% carbon dioxide incubator at a temperature of 37° C. for 24 h;
(4) removing a medium contained in the 6-well plates, washing the B16-F10 cells with 2 mL of the PBS, adding 2 mL of a basal DMEM into each well of one of the 6-well plates to obtain a blank group, adding 1.8 mL of a sample to be tested and 200 μL of the 500 μM α-MSH solution into each well of another one of the 6-well plates to obtain an experimental group, and culturing the blank group and the experimental group in the 5% carbon dioxide incubator at a temperature of 37° C. for 48 h; and
(5) removing the basal DMEM in the 6-well plates by using a sterile pipette, washing the B16-F10 cells with 2 mL of the PBS, digesting the B16-F10 cells with 400 μL of trypsin, terminating the digesting with 1 mL of the complete DMEM, collecting the B16-F10 cells into centrifuge tubes, centrifuging the B16-F10 cells at 2,500 rpm for 10 min, discarding a resulting supernatant after centrifugation, centrifuging the B16-F10 cells with 1,000 μL of the PBS for a second time, adding 500 μL of a 1 mol/L NaOH solution into each of the centrifuge tubes, and conducting water bath at a temperature of 80° C. for 1 h to obtain a product, transferring 200 μL of the product to a 96-well plate, determining an absorbance at 405 nm, and calculating to obtain a relative melanin content.

2. The method of claim 1, wherein the double antibody in step (2) is a penicillin-streptomycin double antibody.

3. The method of claim 1, wherein the inoculating in step (3) is conducted at a cell density of 2×10⁵ cells/mL.

4. The method of claim 1, wherein a concentration of the sample to be tested in step (4) is one or more selected from the group consisting of 0.5 μM, 1.0 μM, 2.5 μM, 5 μM, and 10 μM.

5. The method of claim 1, wherein the method further comprises providing a positive control group in step (4), and the positive control group is set by adding 1.8 mL of β-arbutin and 200 μL of an α-MSH solution into each well of further one of the 6-well plates.

6. The method of claim 1, wherein the method further comprises providing a model group in step (4), and the model group is set by adding 1.8 mL of the sample to be tested with different concentrations and 200 uL of the 500 uM a-MSH solution into each well of further one of the 6- well plates.

7. The method of claim 1, wherein the NaOH solution in step (5) comprises 10% of dimethyl sulfoxide (DMSO).

8. The method of claim 1, wherein a calculation formula of the relative melanin content in step (5) is: the relative melanin content =absorbance of the experimental group/absorbance of the blank group × 100%.

9. A method for using the method of claim 1, comprising using the method in screening the cosmetic raw material with the whitening efficacy.

10. The method of claim 9, wherein the double antibody in step (2) is a penicillin- streptomycin double antibody.

11. The method of claim 9, wherein the inoculating in step (3) is conducted at a cell density of 2×105 cells/mL.

12. The method of claim 9, wherein a concentration of the sample to be tested in step (4) is one or more selected from the group consisting of 0.5 μM, 1.0 μM, 2.5 μM, 5 μM, and 10 μM.

13. The method of claim 9, wherein the method further comprises providing a positive control group in step (4), and the positive control group is set by adding 1.8 mL of β-arbutin and 200 μL of an α-MSH solution into each well of further one of the 6-well plates.

14. The method of claim 9, wherein the method further comprises providing a model group in step (4), and the model group is set by adding 1.8 mL of the sample to be tested with different concentrations and 200 μL of the 500 μM α-MSH solution into each well of further one of the 6-well plates.

15. The method of claim 9, wherein the NaOH solution in step (5) comprises 10% of dimethyl sulfoxide (DMSO).

16. The method of claim 9, wherein a calculation formula of the relative melanin content in step (5) is: the relative melanin content=absorbance of the experimental group/absorbance of the blank group×100%.
